(19) United States
(12) Patent Application Publication
(10) Pub. No.: US 2005/0170378 A1
(43) Pub. Date: Aug. 4, 2005

(54) Methods and systems for joint analysis of array CGH data and gene expression data

(76) Inventors: Zohar H. Yakhini, Ramat Hasharon (IL); Doron Lipson, Tel-Aviv (IL); Amir Ben-Dor, Bellevue, WA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/964,207

(22) Filed: Oct. 12, 2004

Related U.S. Application Data
(60) Provisional application No. 60/541,712, filed on Feb. 3, 2004.

(51) Int. Cl. C12Q 1/68; G06F 19/00; G01N 33/48; G01N 33/50

(52) U.S. Cl. 435/6; 702/20

(57) ABSTRACT

Methods, systems and computer readable media for identifying a high-scoring, significantly altered genomic-continuous submatrix, wherein each genomic-continuous submatrix contains a subset of a set of genes measured across a set of samples to generate a DNA copy number data matrix and a gene expression data matrix. The subset of the genes is a genomic-continuous set of genes, and each genomic-continuous submatrix contains a subset of the set of samples measured to generate the DNA copy number data matrix and the gene expression data matrix.

### CROSS-REFERENCE

This application claims the benefit of U.S. Provisional Application No. 60/541,712, filed Feb. 3, 2004 and titled “Joint Analysis of DNA Copy Numbers and Expression Levels”, which application is incorporated herein by reference, in its entirety.

### BACKGROUND OF THE INVENTION

Alterations in DNA copy number are characteristic of many cancer types and are thought to drive some cancer pathogenesis processes. These alterations include large chromosomal gains and/or losses, as well as smaller scale amplifications and/or deletions.

The mapping of common genomic aberrations has been a useful approach to discovering cancer-related genes. Genomic instability may trigger the over-expression or activation of oncogenes and the silencing of tumor suppressors and DNA repair genes. Local fluorescence in-situ hybridization-based techniques were used early on for measurement of alterations in DNA copy number.

A genome-wide measurement technique referred to as Comparative Genomic Hybridization (CGH) is currently used for identification of chromosomal alterations in cancer, e.g., see Balsara et al., “Chromosomal imbalances in human lung cancer”, Oncogene, 21(45): 6877-83, 2002; and Mertens et al., “Chromosomal imbalance maps of malignant solid tumors: a cytogenetic survey of 3185 neoplasms”, Cancer Research, 57(13): 2765-80, 1997. Using CGH, differentially labeled tumor and normal DNA are co-hybridized to normal metaphases. Ratios between the tumor and normal labels enable the detection of chromosomal amplifications and deletions of regions that may include oncogenes and tumor suppressive genes. This method has a limited resolution however, of only about 10-20 Mbp (mega base pairs). This amount of resolution provided is insufficient to enable a determination of the borders of the chromosomal changes or to identify changes in copy numbers of single genes and small genomic regions.

A more advanced measurement technique referred to as array CGH (aCGH) enables the determination of changes in DNA copy number of relatively small chromosomal regions. Using aCGH, tumor and normal DNA are co-hybridized to a microarray of thousands of genomic clones of BAC, cDNA or oligonucleotide probes, e.g., see Pollack et al., “Genome-wide analysis of dna copy number changes using cdna microarrays”, Nature Genetics, 23(1): 41-6, 1999; Pinkel et al., “High resolution analysis of dna copy number variation using comparative genomic hybridization to microarrays”, Nature Genetics, 20(2): 207-211, 1998; and Hedenfalk et al., “Molecular classification of familial non-brca1/brca2 breast cancer”, PNAS. By using oligonucleotide arrays, the resolution provided can, in theory, be finer than that necessary to identify single genes.

The development of high resolution mapping of DNA copy number alterations and the user of expression profiling technologies have made it possible to study the effects of chromosomal alterations on the cellular processes, as well as to study how the effects are mediated through altered expression of genes residing in altered regions. The measurement of DNA copy numbers and mRNA expression levels with regard to the same set of samples provides information that may reveal the relationship of copy number alterations to how they are manifested in altering expression profiles. Studies that jointly analyze expression and DNA copy number data have, to date, only considered same gene correlations, that is, correlations between the expression levels vector and the DNA copy number vector of the same gene.

Platzer et al., as reported in “Silence of chromosomal amplifications in colon cancer, Cancer Research, 62(4): 1134-8, 2002, used parallel DNA copy number and expression data in metastatic colon cancer samples and concluded that the effect of amplification on increased expression levels is minor. This study did not provide rigorous statistical support for the conclusion, however. For each one of the regions where common amplifications were found, the median expression level of genes that resided in those regions were compared to the median expression levels of the same genes in nine normal control colon samples. A two-fold over-expression was found in eighty-one of the two thousand one hundred forty-six genes that reside in the identified regions. No quantitative statistical analysis of these results was provided, nor were any results for expression fold changes, other than the two-fold results mentioned above, provided. Specific genes in the amplified region that were clearly over-expressed were identified.

Pollack et al., in “Microarray analysis reveals a major direct role of dna copy number alteration in the transcriptional program of human breast tumors”, PNAS, 99(20): 12963-8, 2002, reports an opposite observation regarding breast cancer samples. That is, Pollack et al. report a strong global correlation between copy number changes and expression level variation. Similarly, Hyman et al., in “”Impact of dna amplification on gene expression patterns in breast cancer”, Cancer Research, 62: 6240-5, 2002, studied copy number alterations in fourteen breast cancer cell lines and identified two hundred seventy genes with expression levels that are systematically attributable, in a statistically meaningful manner, to gene amplification. The statistics used by the foregoing studies of Pollack et al. and Hyman et al. were based on simulations and took into account single gene correlations, but not local regional effects.

Linn et al., “Gene expression patterns and gene copy number changes in dfsp”, American Journal of Pathology, 163(6): 2383-2395, 2003, studied expression patterns and genome alterations in DFSP and discovered common 17q and 22q amplifications that are associated with elevated expression of resident genes.

There is a continuing need for methods of statistically supporting data analysis designed to improve the understanding of copy number to transcription relationships. Such need is particularly evident for supporting aCGH data and analysis of the same.

### SUMMARY OF THE INVENTION

Methods, systems and computer readable media are provided for co-analyzing DNA copy number data and gene expression data to identify significant relationships between alterations in genomic DNA and genes that are functionally effected by such alterations. DNA copy number data and gene expression data are provided for a set of genes across a plurality of samples. A gene expression data vector and a DNA copy number data vector is generated for each gene in the set of genes. A gene expression data vector is selected and correlation values are determined between the selected gene expression data vector and DNA copy number vectors corresponding to the selected gene and genes in a defined chromosomal neighborhood of the selected gene, wherein the chromosomal neighborhood includes at least two genes.

Methods, systems and computer readable media are provided for identifying chromosomal regions where consistently biased DNA copy number measurements and corresponding gene expression measurements correlate beyond an extent expected for the consistently biased DNA copy number measurements. A chromosomal neighborhood consisting of a set of loci located about a selected gene is identified. Further, a simulation size is defined by an integer L, and L−1 gene expression vectors are randomly drawn from an expression data matrix having been generated by gene expression data measured across a plurality of samples. A correlation of each randomly drawn gene expression vector to DNA copy number vectors having been generated by DNA copy number data across the plurality of samples for each of the respective genes in the chromosomal neighborhood identified in said identifying step is computed. The computed correlation values computed with respect to the randomly drawn expression vectors are ranked relative to a correlation value computed for the selected gene relative to the neighborhood of DNA copy number vectors, and an indicator of the degree of regional correlation of the DNA copy number vectors from the chromosomal neighborhood to the gene expression vector of the selected gene is calculated.

Methods, systems and computer readable media are provided for detecting chromosomal locations in which genomic aberrations have occurred, samples that are affected by each genomic aberration, and the transcriptional effect of the aberration, based upon co-analysis of DNA copy number data and gene expression data wherein a DNA copy number data matrix provided contains DNA copy number measurements for a set of genes across a set of samples and a gene expression data matrix provided contains gene expression measurements for the same set of genes across the same samples. A genomic-continuous submatrix is identified, containing a subset of the set of genes measured to generate the DNA copy number data matrix and the gene expression data matrix, wherein the subset of the genes is a genomic-continuous set of genes, and wherein the genomic-continuous submatrix contains a subset of the set of samples measured to generate the DNA copy number data matrix and the gene expression data matrix. The DNA copy number data matrix and the gene expression data matrix are projected on the subset of genes and subset of samples and respectively, a DNA copy number data submatrix and a gene express data submatrix corresponding to the genomic-continuous submatrix are generated. The submatrices are scored corresponding to the genomic-continuous submatrix relative to complement DNA copy number data and gene expression data submatrices corresponding to a complement submatrix defined by the same subset of genes in the genomic-continuous submatrix and a complement of the subset of samples in the genomic-continuous submatrix, to determine whether the genomic-continuous submatrix is significantly amplified.

Methods, systems and computer readable media are provided for detecting chromosomal locations in which genomic aberrations have occurred, samples that are affected by each genomic aberration, and the transcriptional effect of the aberration, based upon co-analysis of DNA copy number data and gene expression data wherein a DNA copy number data matrix provided contains DNA copy number measurements for a set of genes across a set of samples and a gene expression data matrix provided contains gene expression measurements for the same set of genes across the same samples. A genomic-continuous submatrix is identified, containing a subset of the set of genes measured to generate the DNA copy number data matrix and the gene expression data matrix, wherein the subset of the genes is a genomic-continuous set of genes, and wherein the genomic-continuous submatrix contains a subset of the set of samples measured to generate the DNA copy number data matrix and the gene expression data matrix. A complement submatrix is identified and defined by the same subset of genes in the genomic-continuous submatrix and a complement of the subset of samples in the genomic-continuous submatrix. The DNA copy number data matrix and the gene expression data matrix are projected on the subset of genes and subset of samples and respectively, a DNA copy number data submatrix and a gene expression data submatrix are generated corresponding to the genomic-continuous submatrix. The submatrices corresponding to the genomic-continuous submatrix relative to DNA copy number data and gene expression data submatrices are scored corresponding to the complement submatrix, to determine whether a significant deletion has occurred in the genomic-continuous submatrix.

Methods, systems and computer readable media are provided for identifying high-scoring, significantly altered genomic-continuous submatrices, wherein each genomic-continuous submatrix contains a subset of a set of genes measured across a set of samples to generate a DNA copy number data matrix and a gene expression data matrix, wherein the subset of the genes is a genomic-continuous set of genes, and wherein each genomic-continuous submatrix contains a subset of the set of samples measured to generate the DNA copy number data matrix and the gene expression data matrix. A continuous segment of genes having a segment length less than or equal to a predefined segment length as the subset of genes is identified, and, for each sample in the set of samples, the DNA copy number data matrix is projected on the sample and the subset of genes and a DNA copy number data column vector is formed corresponding to each sample, respectively. The number of values which are greater than a predetermined threshold value in each of the data column vectors formed is counted, and the samples are ordered according to the counts of the respective DNA copy number vectors. Order prefixes of the set of samples are then scored as to degree of amplification based on overabundance of values greater than the predetermined threshold value in the corresponding DNA copy number submatrices relative to a corresponding complement DNA copy number submatrix containing measurements characterizing the same subset of genes as in the corresponding DNA copy number submatrix, but the complement of the subset of samples characterized in the corresponding DNA copy submatrix. A maximum score is determined from the degree of amplification scores. If the maximum score determined is greater than a predetermined significance threshold, the genomic-continuous submatrix corresponding to the subset of samples from which the maximum score was calculated is concluded to be a significantly amplified genomic-continuous submatrix.

Methods, systems and computer readable media are provided for identifying a high-scoring, significantly altered genomic-continuous submatrices, wherein each genomic-continuous submatrix contains a subset of a set of genes measured across a set of samples to generate a DNA copy number data matrix and a gene expression data matrix, wherein the subset of the genes is a genomic-continuous set of genes, and wherein each genomic-continuous submatrix contains a subset of the set of samples measured to generate the DNA copy number data matrix and the gene expression data matrix. A continuous segment of genes is identified, having a segment length less than or equal to a predefined segment length as the subset of genes. For each sample in the set of samples, the DNA copy number data matrix is projected on the sample and the subset of genes and a DNA copy number data column vector is formed corresponding to each sample, respectively. The number of values which are less than a predetermined threshold value in each of the data column vectors formed is counted. The samples are then ordered according to the counts of the respective DNA copy number vectors, and order prefixes of the set of samples are scored as to degree of deletion based on overabundance of values less than the predetermined threshold value in the corresponding DNA copy number submatrices relative to a corresponding complement DNA copy number submatrix, where the corresponding complement DNA copy number matrix contains measurements characterizing the same subset of genes as in the corresponding DNA copy number submatrix, but the complement of the subset of samples characterized in the corresponding DNA copy submatrix. A maximum score is determined from the degree of deletion scores, and if the maximum score determined is greater than a predetermined significance threshold, it is concluded that that the genomic-continuous submatrix corresponding to the subset of samples from which the maximum score was calculated, is a significantly deleted genomic-continuous submatrix.

The present invention also covers forwarding, transmitting and/or receiving results from any of the methods described herein.

These and other advantages and features of the invention will become apparent to those persons skilled in the art upon reading the details of the methods, systems and computer readable media as more fully described below.

### BRIEF DESCRIPTION OF THE DRAWINGS

FIG. 1 shows a matrix E representing gene expression (GE) values generated from n samples with regard to M genes.

FIG. 2 shows a matrix C representing DNA copy number (DCN) values generated from n samples with regard to M genes.

FIG. 3 shows an example of a randomly permuted matrix E′ wherein the rows of the matrix have been permuted.

FIG. 4 shows an example of a randomly permuted matrix C′, wherein the rows of the matrix have been permuted.

FIG. 5 illustrates quadrants formed when using a separating-crosses scoring methodology.

FIG. 6. illustrates steps that may be taken in performing a simulation analysis to identify chromosomal regions where consistently biased DNA copy number measurements and the corresponding expression levels correlate beyond the extent expected for the consistent copy number values, to evaluate locus-dependent p-values for chromosomal regions.

FIG. 7 shows plots of the cumulative distribution of p-values for various arrangements of a gene dataset.

FIG. 8 is a flow chart showing events that may be carried out in applying a Max-Hypergeometric analysis as described herein.

FIG. 9 is a flow chart showing events that may be carried out in applying Consistent Correlation analysis as described herein.

FIG. 10 illustrates a typical computer system in accordance with an embodiment of the present invention.

### DETAILED DESCRIPTION OF THE INVENTION

Before the present methods, systems and computer readable media are described, it is to be understood that this invention is not limited to particular examples or embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a vector” includes a plurality of such vectors cells and reference to “the gene” includes reference to one or more genes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

A “microarray”, “bioarray” or “array”, unless a contrary intention appears, includes any one-, two- or three-dimensional arrangement of addressable regions bearing a particular chemical moiety or moieties associated with that region. A microarray is “addressable” in that it has multiple regions of moieties such that a region at a particular predetermined location on the microarray will detect a particular target or class of targets (although a feature may incidentally detect non-targets of that feature). Array features are typically, but need not be, separated by intervening spaces. In the case of an array, the “target” will be referenced as a moiety in a mobile phase, to be detected by probes, which are bound to the substrate at the various regions. However, either of the “target” or “target probes” may be the one, which is to be evaluated by the other.

Methods to fabricate arrays are described in detail in U.S. Pat. Nos. 6,242,266; 6,232,072; 6,180,351; 6,171,797 and 6,323,043. As already mentioned, these references are incorporated herein by reference. Other drop deposition methods can be used for fabrication, as previously described herein. Also, instead of drop deposition methods, photolithographic array fabrication methods may be used. Interfeature areas need not be present particularly when the arrays are made by photolithographic methods as described in those patents.

Following receipt by a user, an array will typically be exposed to a sample and then read. Reading of an array may be accomplished by illuminating the array and reading the location and intensity of resulting fluorescence at multiple regions on each feature of the array. For example, a scanner may be used for this purpose is the AGILENT MICROARRAY SCANNER manufactured by Agilent Technologies, Palo, Alto, Calif. or other similar scanner. Other suitable apparatus and methods are described in U.S. Pat. Nos. 6,518,556; 6,486,457; 6,406,849; 6,371,370; 6,355,921; 6,320,196; 6,251,685 and 6,222,664. However, arrays may be read by any other methods or apparatus than the foregoing, other reading methods including other optical techniques or electrical techniques (where each feature is provided with an electrode to detect bonding at that feature in a manner disclosed in U.S. Pat. Nos. 6,251,685, 6,221,583 and elsewhere).

A “gene expression response signature”, “gene expression data vector” or “expression data vector” refers to a vector generated by expression values of the same gene over a number of samples.

The “set of all measured loci” refers to all loci for which measurement data were obtained in a study under investigation.

A “genomic-continuous set of loci” is a subset of the set of all measured loci, such that there is a chromosome such that all members of the subset are exactly the loci that reside in the chromosome and that have genomic positions between some given first and second genomic positions (i.e., between “genomic position a” and “genomic position b”).

A “DNA copy number data vector” or “copy number data vector” refers to a vector generated by DNA copy number values of the same gene over a number of samples.

The term “penetrance” refers to the degree to which the cells in a sample have been affected by the phenomenon being studied. Thus, for example, a tumor cell population in a sample having low penetrance is one in which not all of, or a relatively low percentage of, tumor cells have altered genomes.

The term “prevalence” refers to the degree to which all of the samples in a study have been affected by the phenomenon being studied. Thus, for example, a study showing low prevalence is one in which not all of, or a relatively low percentage of, samples in the study have altered genomes.

When one item is indicated as being “remote” from another, this is referenced that the two items are at least in different buildings, and may be at least one mile, ten miles, or at least one hundred miles apart.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application, are incorporated into this application by reference except insofar as they may conflict with those of the present application (in which case the present application prevails).

The present invention provides methods, systems and computer readable media for identifying genes that show an expression pattern that significantly correlates with a predetermined number of (typically, most) gene DNA copy number measurements in those genes' chromosomal neighborhoods. From the statistical point of view, such a region-based analysis yields much stronger support to copy number to expression correlations, as compared with single gene comparisons of expression values to DNA copy number values.

The present invention further provides systems, methods and computer readable media to statistically assess the resulting correlation values, for whole datasets, and their dependence on regional phenomena.

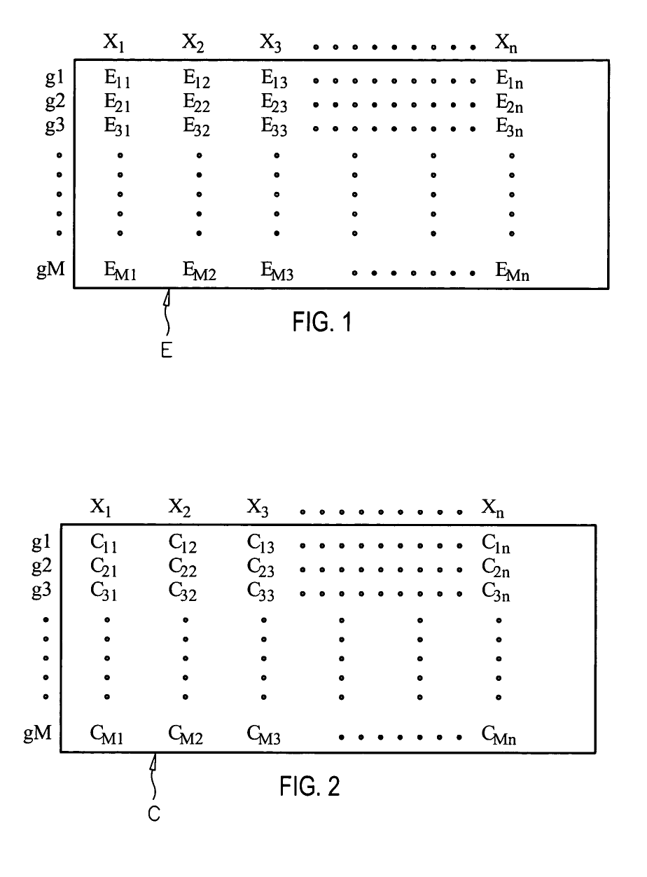

Referring now to FIG. 1, a matrix E of gene expression (GE) values generated from n samples with regard to M genes is shown. For each sample X, the same genes g are measured and expression values are recorded accordingly in matrix E, as values Eᵢⱼ, where the (i,j)^{th} entry of matrix E represents the expression data for the i^{th} gene in the j^{th} sample. For example, expression data value E₂₃ (or, alternatively annotated as E(2,3)) designates the expression value for gene g2 for sample X₃.

Similarly, FIG. 2 shows a matrix C of DNA copy number (DCN) values generated from n samples with regard to M genes. For each sample X, the same genes g are measured for DNA copy number, and DCN values are recorded accordingly in matrix C, as values Cᵢⱼ, where the (i,j)^{th} entry of matrix C represents the DNA copy number data value for the i^{th} gene in the j^{th} sample. For example, DCN data value C₃₃ (or, alternatively annotated as C(3,3)) designates the DCN value for gene g3 for sample X₃. Although the matrices C and E represented in FIGS. 1 and 2 (and the respective microarrays that they represent) contain the same genes (probes), it is noted that the present invention does not require such matrices to contain the same genes (probes). Moreover, DNA copy number matrix C may include entries that correspond to genomic loci that are non-coding.

While, as noted above, matrices C and E may be used to calculate same gene comparisons (e.g., comparing vector E (3, •) with vector C(3, •), where “•” indicates that each column value for the specified row is included in the calculation of the vector, in this example, column values 1 through n, in order to better understand how genome structural instabilities affect cellular processes, and in particular how this effect is mediated through altered expression, it is necessary and useful to analyze chromosomal regions, and not only single genes. Genomic alterations frequently apply to long stretches of the genome that may span a large number of genes. The expression pattern of a gene that is affected by such an aberration is expected to correlate not only with the copy number levels of its own coding DNA, but also with the copy number levels of neighboring genes. Moreover, due to measurement errors, correlation of the measured expression levels of a gene may be stronger when computed against the DNA copy number measured levels of neighboring genes, than when computed against the gene's own DNA copy number measured levels. Accordingly, discussed herein are analysis methods, systems and computer readable media that take regional effects into account to yield better results that may offset the obscuring effects of measurement noise and/or of low prevalence and low penetrance. Low penetrance and/or low prevalence DNA copy number alterations may effect expression below the 2-fold mark, although in a statistically significant manner when regional effects are taken into account.

A region-based analysis, from the statistical point of view, yields much stronger support of copy number to expression corrections, when benchmarked against an appropriately modified null-model. If all the variation in the DNA copy number vector arises due to experimental errors, then the correlation between expression data vectors and their corresponding (same gene, or other gene in the region) DNA copy number data vectors should behave completely randomly.

False Detection Rate (FDR) cutoffs, as discussed in Benjamini et al., “Step-down tests that control the false discovery rate when test statistics are independent”, Journal of Statistical Planning and Inference, 82: 163-70, 1999, which is incorporated herein, in its entirety, by reference thereto, as well as other statistical comparisons are performed to identify genes that reside in aberrant chromosomal regions and produce expression levels that follow a correlated pattern. It has been determined that the analysis of region-based correlations yields many more such correlated genes at a given FDR threshold than an analysis of self-correlation (DNA copy number to expression levels of the same gene).

Correlation Scoring

One of the most common measures of the dependence between two vectors is the Pearson correlation coefficient. The Pearson correlation coefficient measures the dependence between tow vectors, u and v, as follows:

$$r ( u , v ) = \frac{\sum ( u - \overline{u} ) ( v - \overline{v} )}{\sqrt{\sum {( u - \overline{u} )}^{2} \sqrt{\sum {( v - \overline{v} )}^{2}}}} \qquad ( 1 )$$

where r measures the degree to which the two vectors maintain a linear relationship. This correlation metric may therefore be less suitable when the DNA copy number data values and gene expression data values follow some non-linear relationship. Because previous large-scale DCN-GE comparative studies used Pearson correlation as a sole scoring method to evaluate dependence, the significance of the observed Pearson correlation scores are analyzed below using simulations. However, the present invention is not limited to the use of Pearson correlation analysis, as other linear or non-linear correlation metrics may be employed.

A different methodology for comparing gene copy measurements with gene expression levels utilizes user-chosen thresholds for classifying DNA copy number measurements as “deleted” or “amplified”, and further utilizes user-chosen thresholds for classifying gene expression measurements as under-expressed or over-expressed. This approach does not rely upon any assumption of linearity between the DCN measurement vectors and GS measurement vectors, but is somewhat dependent upon the specific choices for thresholds assigned by the user. A generalized approach to threshold-based analysis of the dependence between two vectors is characterized by the separating-crosses scoring methodology described hereafter.

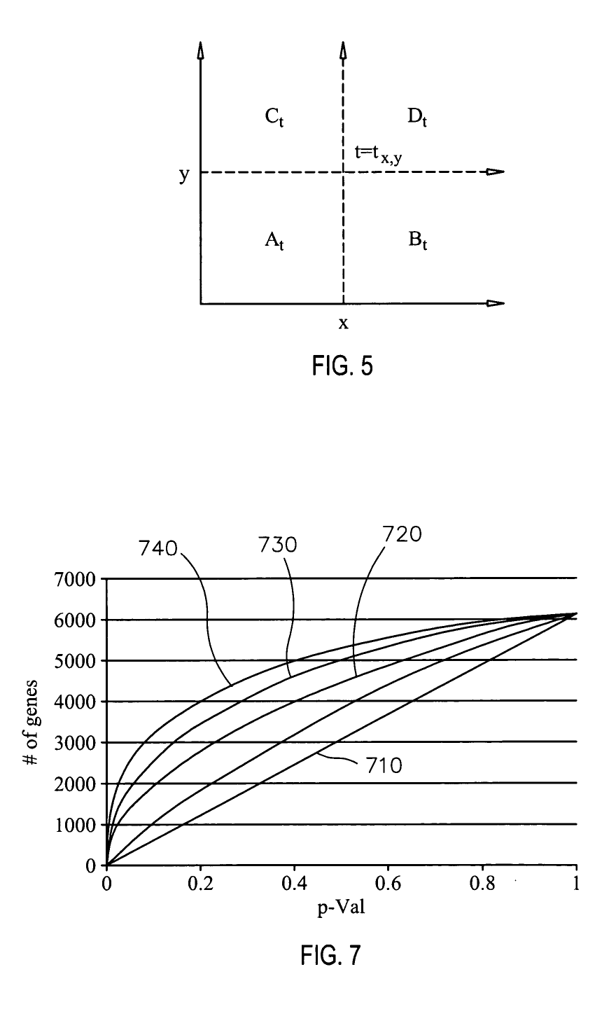

The components of the two vectors u and v are considered as n points (uᵢ,vᵢ) in a plane. An axis parallel cross defined by t=t_{x,y}, centered at (x,y), partitions the plane into four quadrants denoted by Aₜ, Bₜ, Cₜ, and Dₜ, see FIG. 5. The number of points from (uᵢ,vᵢ) that fall in quadrant Aₜ are denoted by aₜ, the number of points from (uᵢ,vᵢ) that fall in quadrant Bₜ are denoted by bₜ, the number of points from (uᵢ,vᵢ) that fall in quadrant Cₜ are denoted by cₜ, and the number of points from (uᵢ,vᵢ) that fall in quadrant Dₜ are denoted by dₜ, such that aₜ+bₜ+cₜ+dₜ=n. The vectors u and v are determined to be correlated if there exists a cross t such that both aₜ and dₜ are large compared to bₜ and cₜ. More generally, given a function of the quadrant counts (i.e., a cross function, f(a,b,c,d), a separating cross score function defines the maximal obtainable value of f, denoted by F, over all possible choices of threshold t. That is:

$$F ( u , v ) = {max}_{t} \{ f ( {a}_{t} , {b}_{t} , {c}_{t} , {d}_{t} ) \} \qquad ( 2 )$$

By ranking the values of the sample in vector u denoted as values of the variable π such that u(π⁻¹(1))<u(π⁻¹(2))< . . . <u(π⁻¹(n)) and by denoting by τ the samples permutation induced by the vector v gives:
F(u,v)=F(π,τ) (3)
since cross-functions, and thus score functions, depend only on the counts of the points in each quadrant and not on the actual locations of the points. Thus, for every function f(π,τ,t), the function F(π,τ) can be computed by examining (n−1)² possible crosses.

A variation of the separating cross score function referred to as the Maximal Diagonal Product (MDP) score considers the separating cross function:
DP(π,τ,t)=aₜ·dₜ (4)
which is also referred to as the Diagonal Product (DP). The corresponding score function of the Diagonal Product, called the Maximal Diagonal Product (MDP is given as follows:

$$MDP ( π , τ ) = {max}_{t} \{ DP ( π , τ , t ) \} \qquad ( 5 )$$

A useful attribute of the MDP score is that it provides a distinction between samples that contribute to the maximum score (i.e., points within quadrants Aₜ and Dₜ) and those that do not (i.e., points within quadrants Bₜ and Cₜ). This attribute is accordingly useful for identifying affected samples versus non-affected samples. The combinatorial nature of this score allows rigorous calculation of its statistical properties.

Another variation of the separating cross score function is called Sum of Diagonal Product (SDP) and is defined by:

$$SDP ( π , τ ) = {\sum }_{t} \{ DP ( π , τ , t ) \} \qquad ( 6 )$$

Regional Analysis

The biological basis for co-analysis of DCN and GE data is the existence of alterations in genomic DNA that have direct effect on mRNA copy number, possibly leading to downstream functional deficiencies. The existence of such alterations is most likely localized in one or more of the following aspects: the alteration in genomic DNA is limited to certain chromosomal segments; the expression of all genes with a specific genomic segment may not be effected to the same extent; not all samples contain identical or similar genomic alterations; and/or within specific samples, a certain alteration may occur with varying levels of penetrance.

As described above, previous studies and analysis using DCN-GE data relationships have considered only correlation between the gene expression levels of single genes and their respective DNA copy number measurements. CGH-based studies show that chromosomal alterations frequently apply to long stretches of the genome that may span a large number of genes. Accordingly, it can be expected that the expression pattern of a gene that is affected by such an aberration will correlate not only with a copy number of its own coding DNA, but also with the DCN measurements of neighboring genes. By applying the principles of the present invention, analysis takes into account regional effects to yield better results that may offset the negative effects of noise in the data or low penetrance of the aberration in some or all samples. Consideration of localized appearances of correlation between genomic alteration and variance in gene expression levels, as described below, account for regional effects of genetic alteration of a gene on its neighboring genes.

Referring again to the expression data and DNA copy number data matrices E and C of FIGS. 1 and 2, ratios, absolute values or logarithmic values may be consistently provided as the member values of these matrices. The Pearson correlation between the vector of DNA copy values of gene gᵢ and the vector of gene expression values of gⱼ may be calculated as follows:

$$r ( i , j ) = Corr ( E ( i , \cdot ) , C ( j , \cdot ) ) = \frac{{\sum }_{k} ( E ( i , k ) - \overline{E ( i , \cdot )} ) ( C ( j , k ) - \overline{C ( j , \cdot )} )}{{{[ {\sum }_{k} {( E ( i , k ) - \overline{E ( i , \cdot )} )}^{2} ]}^{\{ 1 / 2 \}} [ {\sum }_{k} {( C ( j , k ) - \overline{C ( j , \cdot )} )}^{2} ]}^{1 / 2}} \qquad ( 7 )$$

where
r(i,j)=Corr(E(i,•), C(j,•)) is the Pearson correlation coefficient calculated between the i^{th} row of the E matrix (expression data values matrix E) and the j^{th} row of the C matrix (DNA copy number data values matrix C);
E(i,k) is the expression data value in row i, column k of matrix E,
\overline{E(i,•)} is the average expression data value for the i^{th} row of the expression data value matrix E, averaged over all sample values in the row (in the example of FIG. 1, over all sample values 1 through n),
C(j,k) is the DNA copy number data value in row j, column k of matrix C, and
\overline{C(j,•)} is the average DNA copy number data value for the j^{th} row of the DNA copy number data value matrix C, averaged over all sample values in the row (in the example of FIG. 2, over all sample values 1 through n).

The above approach endeavors to identify genes that show an expression pattern that significantly correlates with most gene DNA copy number measurements in the chromosomal neighborhood of the gene identified. A “chromosomal neighborhood” or “k-neighborhood” of a gene is defined as the continuous sequence of genes indexed by
Γₖ(i)=(i−k,i−(k−1), . . . ,i,i+1, . . . ,i+k) (8)
where
Γₖ(i) represents the indexing of the genes in the k-neighborhood of the gene indexed by i, and
k is a predetermined integer used to define the size of the chromosomal neighborhood to be analyzed.

Alternatively, a chromosomal neighborhood may be defined in terms of the physical length of the genomic fragment surrounding a given gene gᵢ, for example, the chromosomal neighborhood may be defined by the gene gᵢ plus 1 Mbp on either side of the gene gᵢ. When defined in this manner, the size of the neighborhood is not constant, in terms of the data that is analyzed with respect to it, but is dependent upon the density (number) of probes that exist in the chromosomal segment so defined as the chromosomal neighborhood.

Using the first approach described above toward defining a chromosomal neighborhood, the chromosomal neighborhood consists of (2k+1) elements (genes). One approach to quantifying the correlation of gene i's expression vector E(i,•) with the DNA copy number vectors in the chromosomal neighborhood Γₖ(i) is to calculate the average correlation of E(i,•) to each of the respective DNA copy number vectors, as follows:

$$r ( i , {Γ}_{k} ( i ) ) = \frac{1}{2 k + 1} {\sum }_{j = i - k}^{i + k} r ( i , j ) \qquad ( 9 )$$

Alternative approaches to regional correlation may consider the correlation of E(i,•) to the vector of weighted or uniform average DNA copy numbers in the neighborhood Γₖ(i), or the product of the p-values of the respective correlations, for example.

Permuted Data

When performing analyses that take gene order into account, analysis results are compared to a null model that assumes that neighboring genes are independent of one another. The null model is a model that contains only normal (non-aberrant) genomic data. With regard to normal (non-aberrant) genomic data, variation in the DNA copy number measurements will arise only due to experimental error and therefore the correlation scores of a given expression vector with the DNA copy number vectors of neighboring loci are expected to be independent.

In the actual genomic data, neighboring genes are not expected to be independent. If genomic aberrations occur, DNA copy number measurements within the altered region are expected to be positively correlated. Also, the correlation score of a given expression vector with the DNA copy number vectors of neighboring loci within the aberration is expected to be positive. That is, if a genomic aberration occurs in a genomic segment, it is expected that the DNA copy numbers and the expression levels of resident loci/genes will be positively correlated. Independence of neighboring genes is assumed only for the null model. Further analyses may be performed on gene-permuted matrices E′ and C′.

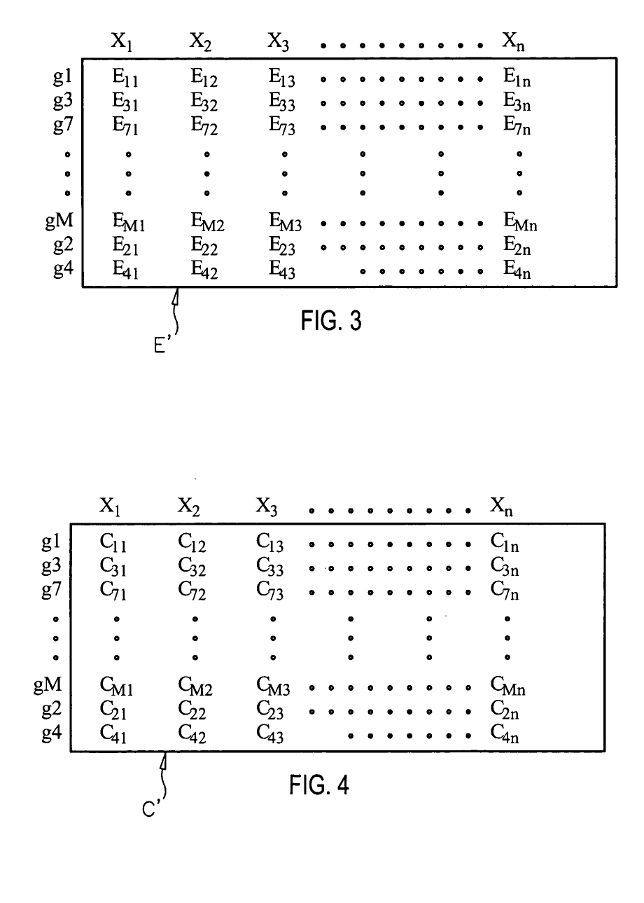

The same permutation is applied to the rows of matrix E as is applied to the rows of matrix C in order to obtain matrices E′ and C′. The rows of data are randomly repositioned in the same manner in each of matrices E and C for each analysis performed. FIGS. 3 and 4 show one non-limiting example of permuted matrices E′ and C′, respectively, where M=k+1 in this example, exhibiting a neighborhood of genes. Since regional effect results are expected to be dependent upon the original chromosomal order of the genes, results for regional effects are corroborated when they diminish greatly upon calculating based on the permuted matrices.

Computing p-Values

A simulation analysis may be performed to identify regions where consistently biased DNA copy number measurements and the corresponding expression levels correlate beyond the extent expected for the consistent copy number values, to evaluate locus-dependent p-values for chromosomal regions. Consistently biased DNA copy number measurements and the corresponding expression levels refer to the expected behavior described above, where DNA copy measurements within an aberrant genomic region are expected to be positively correlated. Correlations in regions where very consistent DNA copy number measurements are observed need to cross much higher thresholds in order to be significant, as compared to correlations in regions where DNA copy number measures are inconsistent, since distributions expected at random in such regions have larger variations. Specifically, there is a relatively weaker smoothing effect of averaging in the case of consistent DNA copy number measurements, due to the consistent DNA copy number values.

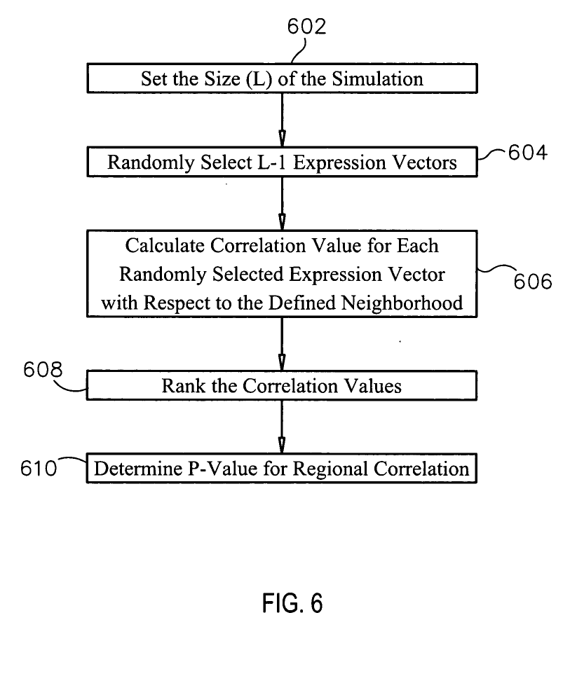

To begin the simulation, the size of the simulation is set as L at event 602, see FIG. 6. The size of the simulation, L, is the amount or number of computations that the researcher is willing (considering time and expense factors, for example) to carry out to get an accurate p-value. For example, an L value of 1000 will yield p-values which are approximately correct down to 0.005, and an L value of 10,000 will yield p-values which are approximately correct down to 0.0005. After setting L, at event 604 L−1 random expression vectors are created or chosen by a user of the system. The random expression vectors can be provided in various manners. For example, L−1 expression vectors may be randomly drawn from matrix E (i.e., rows of matrix E, or, alternatively, L−1 expression vectors may be created using values randomly drawn from matrix E. or randomly drawn from the normal distribution of values, etc. For each randomly drawn expression vector, the correlation of the random expression vector to the neighborhood Γₖ(i) is calculated at event 606 by
rₗ=r(iₗ,Γₖ(i)) (10)

At event 608, the correlation r_{*}=r(i,Γₖ(i)), which is actually observed at i, is assigned a rank ρ amongst r₁,r₂, . . . , r_{L-1}, corresponding to ranks from 1 to L and representing the number of correlation values amongst r₁,r₂, . . . , r_{L-1} and r_{*} that are larger than or equal to r_{*}. At event 610, the p-value for the region correlation observed at i is given by:
pV(i)=ρ/L (11)
where
pV(i) is the p-value for the i^{th} term, and
where the p-value is conditioned on the copy number values of the corresponding chromosomal region.

The above techniques for determining locus dependent p-values were applied to the DCN and GE data values provided in Pollack et al., “Genome-wide analysis of dna copy-number changes using cdna microarrays”, Nature Genetics, 23(1): 41-6, 1999, to investigate copy number to expression correlations. Pollack et al., “Genome-wide analysis of dna copy-number changes using cdna microarrays”, Nature Genetics, 23(1): 41-6, 1999, is hereby incorporated herein, in its entirety, by reference thereto. FIG. 7 shows the cumulative distribution of pV(i), where i ranges over all genes in the dataset. As expected, randomly permuting the dataset yields a straight line 710 that can be used as a reference curve, while significant single gene correlations (i.e., r(i,i), see curve 720) are overabundant at all p-values. Significant correlations are even more abundant when computed for neighborhoods of size k=2 (curve 730) and k=10 (curve 740). Note that these results depend on both the chromosomal order and on direct DCN to GE correlations. Dependence on chromosomal order is evidenced by the fact that the random permutation of the gene data (curve 710) yields a lower abundance of significant correlation scores that singled gene correlations (curve 720). Dependence on direct DCN to GE correlations is represented by the method of calculating pV(i).

The region-dependent pV(i) scores enable the identification of loci where the gene expression levels significantly correlate with the DCN measurements with greater statistical confidence. For example, consider a threshold of 0.001 with regard to the results shown in FIG. 7 (with regard to the data from Pollack et al. referred to above). A random dataset of six thousand genes is expected to contain six genes with this score, whereas single gene correlations yield one hundred sixty four such genes (FDR=3.7%). Considering averaged correlation against Γ₂(i) neighborhoods yields tow hundred fourteen significant loci (FDR=2.8%), and considering averaged correlation against Γ₁₀(i) neighborhoods yields two hundred eighty nine significant loci (FDR=2.1%). Thus, region-based analysis delivers almost eighty percent more loci where GE to DCN correlation may be identified with high confidence.

Genomic-Continuous Submatrices

As noted above, genomic alterations are often localized to a subset of the samples as well as to a specific chromosomal segment of the chromosomal material of those samples affected. The following description addresses the detection of the genomic segment in which an aberration has occurred, the samples that have been affected, and the transcriptional effect of the aberration.

For a given pair of DCN and GE matrices C and E, respectively, over an ordered set of genes G and a set of samples X, a genomic-continuous submatrix (GCSM) can be defined as:
M=G′xX′ (12)
where
M is the GCSM,
G′⊂G and is a continuous segment of genes, and
X′⊂X (X′ is a subset of X up to and including the full set X).

The complement submatrix of the GCSM is defined as:
\overline{M}=G′x(X−X′) (13)
C(M) and E(M) denote the projections of the matrices C and E on the subsets G′ and X′(i.e., the DCN and GE submatrices corresponding to M).

A genomic alteration in a given chromosomal segment and a given sample should affect most of the DNA copy measurements in the given chromosomal segment, but only some of the respective gene expression measurements (i.e., less than the number of affected DNA copy measurements). This is due to the fact that the DCN of any resident gene in the segment is directly affected by the aberrant segment, while the GE of a resident gene may or ay not be modified depending upon different factors that determine regulation of that gene. It is determined that a GCSM M is significantly amplified when most DNA copy values in the set C(M) are positive and some genes Gᵢ∈G′ have higher expression values {E(i,j):Xⱼ∈X′} comparatively to those that are not in the GCSM {E(i,j):Xⱼ∉X′}. The terms “most” and “some” are used informally to convey the qualitative event that is sought to be identified. Examples of formal probabilistic definitions of these events are described below, wherein a hypergeometric or binomial distribution may be used to define the p-value of the overabundance of positive values in C and TNoM binomial surprise analysis may be carried out to define the p-value of the overabundance of good separators in E.

A scoring mechanism that measures the degree to which M has been significantly amplified follows. A score F(M; C) is defined to reflect the overabundance of positive values in C(M) as compared to C(\overline{M}) using the hypergeometric distribution. F is the hypergeometric cumulative distribution function given by:

$$F ( x , M , K , m ) = \frac{{\sum }_{y = 0}^{x} ( \begin{matrix}m \\ y\end{matrix} ) ( \begin{matrix}M - m \\ K - y\end{matrix} )}{( \begin{matrix}M \\ K\end{matrix} )} \qquad ( 14 )$$

The hypergeometric distribution function represents the probability that in drawing objects without replacement from a collection of K black objects and M-K white objects, x or less out of the m objects first drawn are black.

Applying the hypergeometric distribution function to the score F(M; C), let N=|C(M∪\overline{M})| and n=|C(M)|. Further, let K be the number of positive values in C(M∪\overline{M}) and k be the number of positive values in C(M). Given N, n, K, the hypergeometric probability of finding k or more positive values in C(M) is:

$$F ( M ; C ) = HG ( N , K , n , k ) = {\sum }_{i = k}^{N} \frac{( \begin{matrix}n \\ i\end{matrix} ) ( \begin{matrix}N - n \\ K - 1\end{matrix} )}{( \begin{matrix}N \\ K\end{matrix} )} \qquad ( 15 )$$

Alternatively, the overabundance of positive values in C(M) may be assessed using binomial surprise analysis of the fraction of positive values in C(M), given the fraction of positive values in the complete matrix C. The binomial surprise analysis may be carried out using the binomial tail probability of encountering at least the observed number of positive values in C(M), given the fraction of positive values in the complete matrix C.

Similarly, a score function F(M; E) is defined to reflect the overabundance of genes in g′ that are significantly differentially expressed when comparing the expression values in X and X′, i.e., identifying expression levels in X′ that are significantly higher than those in X−X′. A TNoM (Threshold Number of Misclassifications) score may be assigned to each gene according to its performance as an X′ versus an X−X′ classifier.

The TNoM score is based on searching for a simple rule that uses a given expression level, for the given gene, to predict the label of an unknown. Formally, a rule is defined by two parameters a, and b. The predicted class is simply sign(ax+b). Since only the sign of the linear expression matters, attention can be limited to a ∈{−1,+1}. A natural approach is to choose the values of a and b to minimize the number of errors:

$$Err ( a , b \vert g ) \text{=≤} {\sum }_{i} 1 \{ {l}_{i} \neq sign ( a \cdot {x}_{i} [ G ] + b ) \} \qquad ( 16 )$$

where xᵢ[g] is the expression value of gene g in the i^{th} sample. The best values are found by exhaustively trying all 2(m+1) possible rules. Attention is limited to threshold values that are mid-way points between actual expression values.

The TNoM score of a gene is defined as:

$$TNoM ( G ) = {min}_{a , b} Err ( a , b \vert g ) \qquad ( 17 )$$

and defines the number of errors made by the best rule. The intuition is that this number reflects the quality of decisions made based solely on the expression levels of this gene. A further detailed description of the TNoM score and its applications can be found in co-pending, commonly assigned application Ser. No. 10/817,244 filed Apr. 3, 2004 and titled “Visualizing Expression Data on Chromosomal Graphic Schemes”. Application Ser. No. 10/817,244 is hereby incorporated herein, in its entirety, by reference thereto.

Rigorous p-values can be calculated for TNoM scores. If the probability for a single gene, of obtaining a score of s or better under the null model is p(s), then the number of genes with scores of s or better, amongst the |g′| genes examined is binomially distributed (n, p(s)). Letting n(s) denote the number of genes with scores of s or better that are actually observed in the data, and σ(s) denote the tail probability of the binomial (n, p(s)) distribution at n(s), then F(M;E) is defined to be max_{0≦s≦|X′|}−log(σ(s)).

According to the null model, the DCN and GE vectors are completely uncorrelated. A total score for an amplification in M is given by:
F(M; C, E)=−[log₁₀F(M; C)+log₁₀F(M; E)] (18)
It should be noted that the above analysis is not limited to addressing amplifications of genetic material, but is also addresses deletions. Any deletion in a subset X′ is equivalent, under F, to an amplification in X−X′.

Locating Partitions that Yield High-Scoring, Significantly Altered GCSMs

The task of locating a partition of samples that maximizes TNoM overabundance for a given set of genes is by itself a difficult task that has been approached using heuristic methods. The task of location a partition that maximizes a combined hypergeometric and TNoM overabundance score is clearly at least as difficult, and consequently, heuristic methods are applied here for locating significantly altered GCSMs. Since it is important to look for continuous segments of genes only, all possible segments may be enumerated in O(n²), where the term “O” denotes an upper bound on the complexity (or running time) of an algorithm on a computer system, and where n is the number of genes in the dataset. For example, if an algorithm runs in O(f(n)) time, this means that for all n>n₀, the running time of the algorithm is less that c*f(n) for some constants n₀ and c. A difficult task is determining which partition X′, out of the possible 2^{|X|} partitions, maximizes the significance score X((G′xX′); C, E) for a given segment G′. Two approaches are described in the following for locating partitions that yield high-scoring significantly altered GCSMs.

The first approach employs what we refer to as the Max-Hypergeometric Algorithm. Since the definition of the score of a GCSM M is composed of two parts (i.e., hypergeometric part and TNoM part), this approach to locating high-scoring GCSMs selects the sample partitions that maximize one part of the score, in this case the hypergeometric score, for each possible segment, and then calculates the combined scores for those selected. For a given segment G′, the calculation of max_{X′⊂X}[−log(F((G′xX′); C)] may be performed in (O(|X|)) time (and thus, the running time of the algorithm is linearly proportional to the number of elements in X) as follows: let pᵢ equal the number of positive entries in the vector C(G′,sᵢ). Next, the samples are reordered so that P_{π(1)}≧p_{π(2)}≧ . . . ≧p_{n|X|}. The subset X′ that maximizes the score [−log(F((G′xX′);C] is one of the subsets in the collection {(S_{π(1)}),(s_{π(1)},s_{π(2)}), . . . ,(s_{π(1)},s_{π(2)}, . . . ,s_{π(|X|-1)})}.

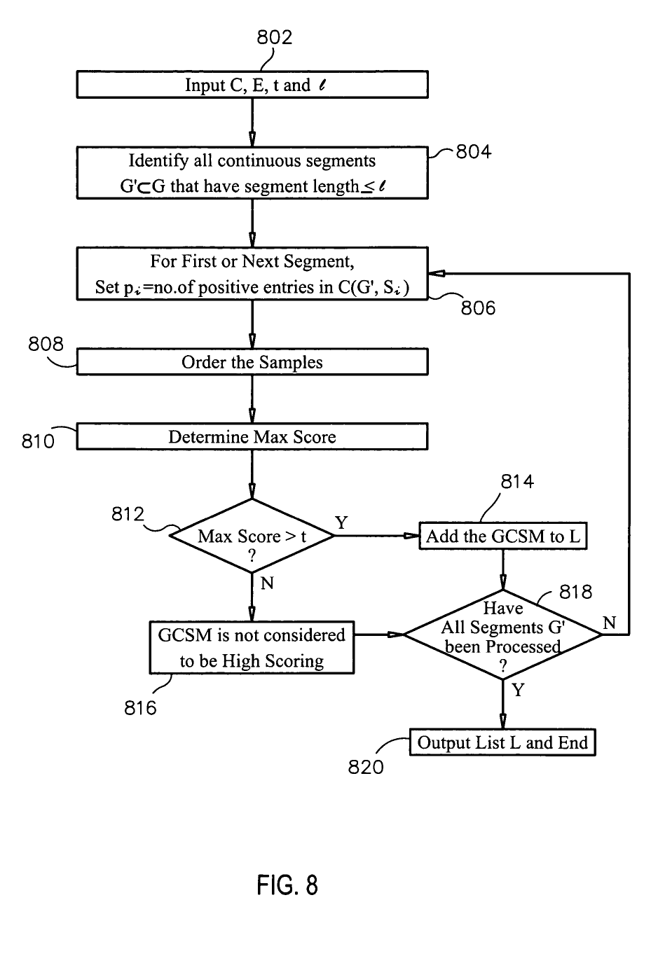

Referring now to FIG. 8, a flow chart of events that may be carried out in applying the Max-Hypergeometric analysis is shown. At event 802, the matrices C and E are inputted, as well as a value for the variable t, which designates a significance threshold, and a value for l, which sets the maximum segment length. At event 804, all segments G′⊂G are identified that have a segment length less than or equal to l. As noted earlier, all segments identified must be continuous segments. At event 806 for the first or next identified segment, pᵢ is set to equal the number of positive entries in C(G′,sᵢ). At event 808, the samples are ordered such that p_{π(1)}≧p_{π(2)}≧ . . . ≧p_{π|X|}. The maximum score is determined at event 810 according to the following:
max Score=max_{1≦i<|X|}F((G′,{s_{π(1)}, . . . ,s_{π(i)}});C,E) (19)
At event 812 it is determined whether the maximum score is greater than the significance threshold. If max Score>t, then the GCSM currently defined is added to L at event 814 (i.e., add M=(G′xX′) to L), which is a list of high scoring GCSMs that is outputted by the process/system. Otherwise, the current GCSM is not considered to be a high-scoring, significantly altered GCSM at event 816.

If all the identified segments have been processed according to events 806-816, as determined at event 818, then list L is outputted by the system (to a user interface, storage device and/or printed out) and processing ends at event 820. Otherwise, processing returns to event 806 to work with the next identified segment.

One shortcoming of the Max-Hypergeometric approach described above is that it depends on a sufficiently strong pattern in the DCN measurements alone in order to detect high-scoring, significantly altered GCSMs. However, in some cases, significant correlation between DCN and GE patterns is indicative of a chromosomal aberration even when the DCN signal by itself is weak. The next technique described for identifying high-scoring, significantly altered GCSMs relies on DCN-GE correlations for location candidate partitions (X′) for a given segment G′, which segments are expected to yield high-scoring GCSMs.

This approach makes use of a helpful attribute of the MDP correlation score described above. That is, for a given gene gᵢ the score MDP(i) defines a cross-threshold t that separates the |X| samples into quadrants such that the product Aₜ·Dₜ is maximized. Hence the samples that contribute to the score MDP(i) (i.e., those that lie within Aₜ or Dₜ) can be readily separated from those that do not contribute to the score (i.e., those that lie within Bₜ or Cₜ). Taking into account the chromosomal neighborhood of gene gᵢ, one can increase confidence that the expression level of gᵢ in a specific sample is affected by the aberration.

For example, assuming that for all correlations of E(i) against Γₖ(i), the same sample s falls in quadrant Dₜ of the respective MDP cross-thresholds. The probability of such an event occurring by chance decreases exponentially with k, the size of the neighborhood. For a gene gᵢ and a sample s∈X, the Sample MDP Score (SMDP) is therefore defined as:

$$SMDP ( s , i ) = \frac{1}{2 k + 1} {\sum }_{j = i - k}^{i + k} \{ [ {1}_{s ∈ {A}_{t} ( i , j )} MDP ( i , j ) ] - [ {1}_{s ∈ {D}_{t} ( i , j )} MDP ( i , j ) ] \} \qquad ( 20 )$$

where Aₜ(i,j) and Dₜ(i,j) are the sets of samples that fall into quadrants Aₜ and Dₜ, respectively, for the threshold t that yields the maximum MDP score for the vectors E(i) and C(j). Note that
−MDP(i,Γₖ(i))≦SMDP(s,i)≦MDP(i,Γₖ(i)) (21)
and extrema are attained if s falls in either quadrant Aₜ or quadrant Dₜ in all of the crosses.

This technique provides for the ranking of the set of samples s∈X according to increasing probabilities that they have been affected by an alteration (amplification/deletion). This ranking suggests O(|X|) possible partitions that should be evaluated. In practice, processing may be run on a filtered set of genes \tilde{G}⊂G that pass some minimal regional correlation threshold, in accordance with the statistical results from regional analysis processing described above.

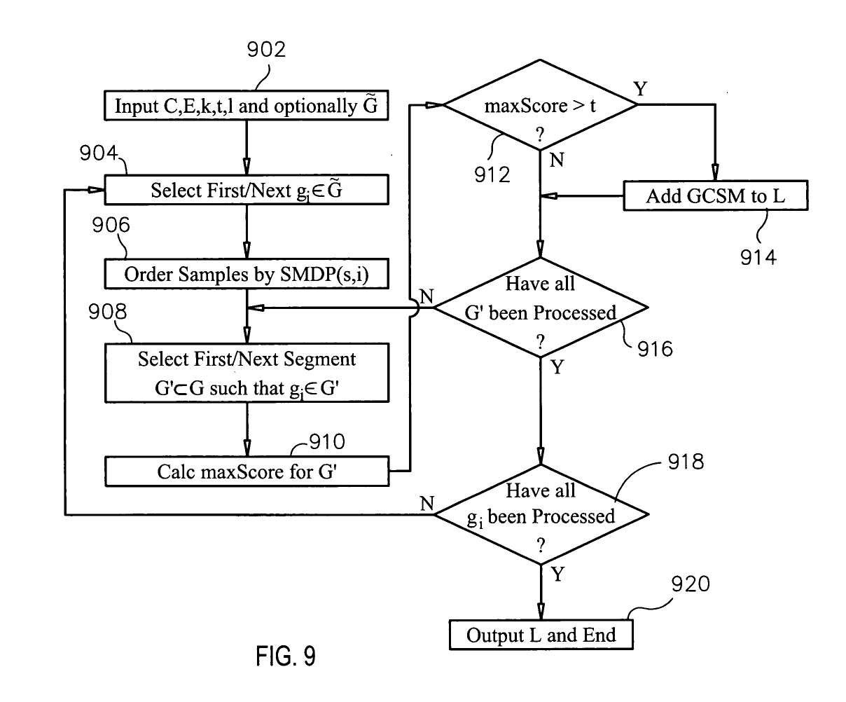

Referring now to FIG. 9, a flow chart of events that may be carried out in applying Consistent Correlation analysis, as described above, is shown. At event 902, the matrices C and E are inputted, as well as optionally inputting a filtered set of genes \tilde{G} to be analyzed if it is not desired to analyze all genes represented by matrices C and E (as described above), a value for k to define the neighborhood size, a value for t to define a significance threshold, and a value for l, which sets the maximum segment length. At event 904, a gene is selected from the set of genes (G or \tilde{G}, as the case may be), and SMDP scores are calculated with regard to each sample sⱼ∈X, with respect to the selected gene. Scores are calculated as follows: pᵢ=SMDP(sⱼ,i). At event 906, the samples are ordered such that p_{π(1)}≧p_{π(2)}≧ . . . ≧p_{π|X|}. A first or next segment (continuous segment) G′⊂G that has a length less than or equal to l, such that gᵢ∈G′ is selected at event 908, and a maximum score is calculated at event 910 as follows:
max Score=max_{1≦i≦|X|}F((G′,{X_{π(1)}, . . . ,X_{π(i)}});C,E) (19)

At event 912 it is determined whether the maximum score is greater than the significance threshold. If max Score>t, then the GCSM currently defined is added to L at event 914 (i.e., add M=(G′xX′) to L), a list of high scoring GCSMs that is outputted by the system. (Although, this example is described with identification of significant amplifications, significant deletions may be identified by a similar process. For example, when considering deletions, the GCSM is added to L when the GCSM score exceeds a significance threshold.) Otherwise, the current GCSM is not considered to be a high-scoring, significantly altered GCSM at event 912, and is not added to list L.

In either case, after the determination is made at event 912 whether to add the current GCSM to the list L, at event 916 a check is made to determined whether all segments G′ have been processed with regard to the currently selected gene gᵢ. If all the identified segments G′ have not yet been processed with respect to the currently selected gene, then processing returns to event 908 to select and process the next identified segment.

If all the identified segments have been processed with regard to the currently selected gene, according to events 908-914, as determined at event 916, then, at event 918, it is determined whether all genes from the set (G or \tilde{G}, as the case may be) have been processed. If all genes gᵢ have yet been processed, then processing returns to event 904, where the next gene gᵢ from the set is selected for processing, and processing continues to event 906 in the manner described above. If, on the other hand, it is determined that all genes gᵢ have been processed, then list L is provided/outputted by the system (to a user interface, storage device and/or printed out) and processing ends at event 920.

The Max-Hypergeometric technique and the Consistent Correlation technique described above are appropriate for cases of high-scoring GCSMs with differing biological motivations. The Max-Hypergeometric technique is better when F(M; C) is a dominant factor of the total score, that is when DCN measurements alone contain a significant pattern due to a chromosomal aberration. The Consistent Correlation technique is appropriate when there is a strong correlation between E(M) and C(M) suggesting that both F(M; C) and F(M;E) have significant influence on the total score. This situation may arise when a chromosomal alteration has significant effect on transcriptional activity.

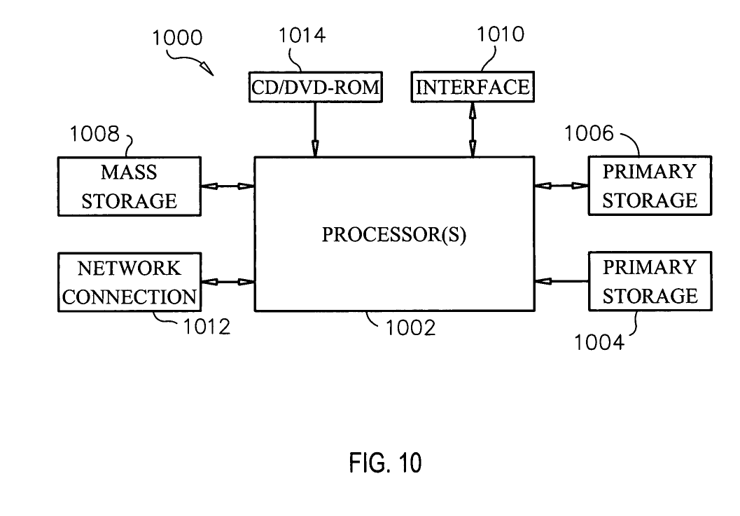

FIG. 10 illustrates a typical computer system in accordance with an embodiment of the present invention. The computer system 1000 includes any number of processors 1002 (also referred to as central processing units, or CPUs) that are coupled to storage devices including primary storage 1006 (typically a random access memory, or RAM), primary storage 1004 (typically a read only memory, or ROM). As is well known in the art, primary storage 1004 acts to transfer data and instructions uni-directionally to the CPU and primary storage 1006 is used typically to transfer data and instructions in a bi-directional manner Both of these primary storage devices may include any suitable computer-readable media such as those described above. A mass storage device 1008 is also coupled bi-directionally to CPU 1002 and provides additional data storage capacity and may include any of the computer-readable media described above. Mass storage device 1008 may be used to store programs, data and the like and is typically a secondary storage medium such as a hard disk that is slower than primary storage. It will be appreciated that the information retained within the mass storage device 1008, may, in appropriate cases, be incorporated in standard fashion as part of primary storage 1006 as virtual memory. A specific mass storage device such as a CD-ROM or DVD-ROM 1014 may also pass data uni-directionally to the CPU.

CPU 1002 is also coupled to an interface 1010 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1002 optionally may be coupled to a computer or telecommunications network using a network connection as shown generally at 1012. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for population of stencils may be stored on mass storage device 1008 or 1014 and executed on CPU 1008 in conjunction with primary memory 1006.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floptical disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of co-analyzing DNA copy number data and gene expression data to identify significant relationships between alterations in genomic DNA and genes that are functionally effected by such alterations, said method comprising the steps of:
providing DNA copy number data and gene expression data for a set of genes across a plurality of samples;
generating a gene expression data vector and a DNA copy number data vector for each gene in the set of genes:
selecting a gene expression data vector; and
determining correlation values between the selected gene expression data vector and DNA copy number vectors corresponding to the selected gene and genes in a defined chromosomal neighborhood of the selected gene, wherein the chromosomal neighborhood includes at least two genes.

2. The method of claim 1, wherein the defined chromosomal neighborhood is a genomic-continuous set of genes.

3. The method of claim 1, wherein the defined chromosomal neighborhood is a k-neighborhood defined b of genes consisting of (2k+1) genes indexed by:
Γₖ(i)=(i−k, i−(k−1), . . . ,i,i+1, . . . ,i+k) (8)
where Γₖ(i) represents the indexing of the genes in the k-neighborhood of the selected gene indexed by i, and
k is a predetermined integer used to define the size of the chromosomal neighborhood to be analyzed.

4. The method of claim 1, wherein said determining correlation values comprises calculating an average correlation of the selected gene expression data vector to each of the respective DNA copy number vectors corresponding to the selected gene and the genes in the defined chromosomal neighborhood.

5. The method of claim 1, wherein said determining correlation values comprises calculating a correlation of the selected gene expression data vector to a vector of weighted or uniform average DNA copy number calculated from the DNA copy number vectors corresponding to the selected gene and the genes in the defined chromosomal neighborhood.

6. The method of claim 1, wherein said determining correlation values comprises calculating the product of p-values of respective correlations of the selected gene expression data vector to each of the respective DNA copy number vectors corresponding to the selected gene and the genes in the defined chromosomal neighborhood.

7. The method of claim 1, further comprising comparing the determined correlation values to correlation values generated from a null model.

8. The method of claim 7, wherein the null model is generated by randomly permuting the order of genes in the same manner in each of the DNA copy number and gene expression datasets, and wherein the correlation values are generated from the null model according to said generating, selecting and determining steps, wherein the same gene expression data vector is selected in the null model as was selected in the method of claim 1.

9. A method comprising forwarding a result obtained from the method of claim 1 to a remote location.

10. A method comprising transmitting data representing a result obtained from the method of claim 1 to a remote location.

11. A method comprising receiving a result obtained from a method of claim 1 from a remote location.

12. A method of identifying chromosomal regions where consistently biased DNA copy number measurements and corresponding gene expression measurements correlate beyond an extent expected for the consistently biased DNA copy number measurements, said method comprising the steps of:
identifying a chromosomal neighborhood consisting of a set of loci located about a selected gene;
defining a simulation size by an integer L;
randomly drawing L−1 gene expression vectors from an expression data matrix having been generated by gene expression data measured across a plurality of samples;
computing a correlation of each randomly drawn gene expression vector to DNA copy number vectors having been generated by DNA copy number data across the plurality of samples for each of the respective genes in the chromosomal neighborhood identified in said identifying step;
ranking the computed correlation values computed with respect to the randomly drawn expression vectors, relative to a correlation value computed for the selected gene relative to the neighborhood of DNA copy number vectors; and
calculating an indicator of the degree of regional correlation of the DNA copy number vectors from the chromosomal neighborhood to the gene expression vector of the selected gene.

13. The method of claim 12, wherein said calculating an indicator comprises calculating a p-value.

14. The method of claim 12, wherein the p-value is defined by the rank of the DNA copy number vector amongst all L vectors divided by L.

15. A method of detecting a chromosomal location in which a genomic aberration has occurred, samples that are affected by the genomic aberration, and the transcriptional effect of the aberration, based upon co-analysis of DNA copy number data and gene expression data wherein a DNA copy number data matrix provided contains DNA copy number measurements for a set of genes across a set of samples and a gene expression data matrix provided contains gene expression measurements for the same set of genes across the same samples, said method comprising the steps of:
identifying a genomic-continuous submatrix containing a subset of the set of genes measured to generate the DNA copy number data matrix and the gene expression data matrix, wherein the subset of the genes is a genomic-continuous set of genes, and wherein the genomic-continuous submatrix contains a subset of the set of samples measured to generate the DNA copy number data matrix and the gene expression data matrix;
projecting the DNA copy number data matrix and the gene expression data matrix on the subset of genes and subset of samples and respectively generating a DNA copy number data submatrix and a gene express data submatrix corresponding to the genomic-continuous submatrix; and
scoring the submatrices corresponding to the genomic-continuous submatrix relative to complement DNA copy number data and gene expression data submatrices corresponding to a complement submatrix defined by the same subset of genes in the genomic-continuous submatrix and a complement of the subset of samples in the genomic-continuous submatrix, to determine whether the genomic-continuous submatrix is significantly amplified.

16. The method of claim 15, wherein the genomic-continuous submatrix is determined to be significantly amplified when a statistically significant proportion of DNA copy number values in the DNA copy number data submatrix corresponding to the genomic-continuous submatrix are greater than a predefined threshold value and some gene expression values in the gene expression data submatrix corresponding to the enomic-continuous submatrix are higher than corresponding gene expression values in the complement gene expression data submatrix.

17. The method of claim 16, wherein said predefined threshold value is zero.

18. The method of claim 15, wherein said scoring comprises scoring the overabundance of values that are greater than a predefined threshold value in the DNA copy number data submatrix relative to the number of values that are greater than the predefined threshold value in the complement DNA copy number data submatrix using a hypergeometric distribution function.

19. The method of claim 18, wherein the predefined threshold value is zero.

20. The method of claim 15, wherein said scoring comprises scoring the overabundance of values that are greater than a predefined threshold value in the DNA copy number data submatrix relative to the number of values that are greater than the predefined threshold value in the entire DNA copy number data matrix using a binomial distribution function.

21. The method of claim 20, wherein the predefined threshold value is zero.

22. The method of claim 15, wherein said scoring comprises scoring the overabundance of values that are greater than a predefined threshold value in the DNA copy number data submatrix relative to the number of values that are greater than the predefined threshold value in the entire DNA copy number data matrix using a normal distribution function.

23. The method of claim 22, wherein the predefined threshold value is zero.

24. The method of claim 15, wherein said scoring comprises scoring the overabundance of genes in the subset of genes that have higher expression values for samples in the data submatrix than for samples in the complement data submatrix.

25. The method of claim 24, wherein said scoring comprises assigning a TNoM score to each gene in the subset of genes indicating its performance as a classifier of the subset of samples versus the complement of the subset of samples.

26. A method of detecting a chromosomal location in which a genomic aberration has occurred, samples that are affected by the genomic aberration, and the transcriptional effect of the aberration, based upon co-analysis of DNA copy number data and gene expression data wherein a DNA copy number data matrix provided contains DNA copy number measurements for a set of genes across a set of samples and a gene expression data matrix provided contains gene expression measurements for the same set of genes across the same samples, said method comprising the steps of:
identifying a genomic-continuous submatrix containing a subset of the set of genes measured to generate the DNA copy number data matrix and the gene expression data matrix, wherein the subset of the genes is a genomic-continuous set of genes, and wherein the genomic-continuous submatrix contains a subset of the set of samples measured to generate the DNA copy number data matrix and the gene expression data matrix;
identifying a complement submatrix defined by the same subset of genes in the genomic-continuous submatrix and a complement of the subset of samples in the genomic-continuous submatrix;
projecting the DNA copy number data matrix and the gene expression data matrix on the subset of genes and subset of samples and respectively generating a DNA copy number data submatrix and a gene expression data submatrix corresponding to the genomic-continuous submatrix; and
scoring the submatrices corresponding to the genomic-continuous submatrix relative to DNA copy number data and gene expression data submatrices corresponding to the complement submatrix, to determine whether a significant deletion has occurred in the genomic-continuous submatrix.

27. The method of claim 26, wherein a significant deletion in the genomic-continuous submatrix is determined to have occurred when a statistically significant proportion of DNA copy number values in the DNA copy number data submatrix corresponding to the genomic-continuous submatrix are less than a predefined threshold value and some gene expression values in the gene expression data submatrix corresponding to the genomic-continuous submatrix are lower than corresponding gene expression values in the complement gene expression data submatrix.

28. The method of claim 27, wherein said predefined threshold value is zero.

29. The method of claim 26, wherein said scoring comprises scoring the overabundance of values less than a predefined value in the DNA copy number data submatrix relative to the number of values less than the predefined value in the complement DNA copy number data submatrix using a hypergeometric distribution function.

30. The method of claim 29, wherein said predefined threshold value is zero.

31. The method of claim 26, wherein said scoring comprises scoring the overabundance of values less than a predefined value in the DNA copy number data submatrix relative to the number of values less than the predefined value in the entire DNA copy number data matrix using a binomial distribution function.

32. The method of claim 31, wherein said predefined threshold value is zero.

33. The method of claim 26, wherein said scoring comprises scoring the overabundance of values less than a predefined value in the DNA copy number data submatrix relative to the number of values less than the predefined value in the entire DNA copy number data matrix using a normal distribution function.

34. The method of claim 32, wherein said predefined threshold value is zero.

35. The method of claim 26, wherein said scoring comprises scoring the overabundance of genes in the subset of genes that have lower expression values for samples in the data submatrix than for samples in the complement data submatrix.

36. The method of claim 35, wherein said scoring comprises assigning a TNoM score to each gene in the subset of genes indicating its performance as a classifier of the subset of samples versus the complement of the subset of samples.

37. A method of identifying a high-scoring, significantly altered genomic-continuous submatrix, wherein each genomic-continuous submatrix contains a subset of a set of genes measured across a set of samples to generate a DNA copy number data matrix and a gene expression data matrix, wherein the subset of the genes is a genomic-continuous set of genes, and wherein each genomic-continuous submatrix contains a subset of the set of samples measured to generate the DNA copy number data matrix and the gene expression data matrix, said method comprising the steps of:
identifying a continuous segment of genes having a segment length less than or equal to a predefined segment length as the subset of genes;
for each sample in the set of samples, projecting the DNA copy number data matrix on the sample and the subset of genes and forming a DNA copy number data column vector corresponding to each sample, respectively;
counting the number of values which are greater than a predetermined threshold value in each of the data column vectors formed;
ordering the samples according to the counts of the respective DNA copy number vectors;
scoring order prefixes of the set of samples as to degree of amplification based on overabundance of values greater than the predetermined threshold value in the corresponding DNA copy number submatrices relative to a corresponding complement DNA copy number submatrix containing measurements characterizing the same subset of genes as in the corresponding DNA copy number submatrix, but the complement of the subset of samples characterized in the corresponding DNA copy submatrix;
determining the maximum score from the degree of amplification scores; and
if the maximum score determined is greater than a predetermined significance threshold, concluding that the genomic-continuous submatrix corresponding to the subset of samples from which the maximum score was calculated, is a significantly amplified genomic-continuous submatrix.

38. The method of claim 37, wherein said predetermined threshold value is zero.

39. The method of claim 37, further comprising identifying all continuous segments of genes having a segment length less than or equal to the predefined segment length; and repeating said projecting, forming, scoring the DNA copy number submatrices, ordering the samples, scoring the ordered samples, determining the maximum score and concluding steps for each of the identified, continuous segments.

40. The method of claim 39, further comprising providing results identifying all genomic-continuous submatrices that were concluded to be significantly amplified.

41. The method of claim 37, wherein said order prefixes are scored according to the hypergeometric distribution function.

42. The method of claim 37, wherein said order prefixes are scored using a binomial distribution function to score the overabundance of values greater than the predetermined threshold value in the DNA copy number data submatrix relative to the number of values greater than the predetermined threshold value in the entire DNA copy number data matrix.

43. The method of claim 37, wherein said order prefixes are scored using a normal distribution function to score the overabundance of values greater than the predetermined threshold value in the DNA copy number data submatrix relative to the number of values greater than the predetermined threshold value in the entire DNA copy number data matrix.

44. The method of claim 37, wherein said scoring comprises scoring the overabundance of genes in the subset of genes that have higher expression values for samples in the data submatrix than for samples in the complement data submatrix.

45. The method of claim 44, wherein said scoring comprises assigning a TNoM score to each gene in the subset of genes indicating its performance as a classifier of the subset of samples versus the complement of the subset of samples.

46. A method of identifying a high-scoring, significantly altered genomic-continuous submatrix, wherein each genomic-continuous submatrix contains a subset of a set of genes measured across a set of samples to generate a DNA copy number data matrix and a gene expression data matrix, wherein the subset of the genes is a genomic-continuous set of genes, and wherein each genomic-continuous submatrix contains a subset of the set of samples measured to generate the DNA copy number data matrix and the gene expression data matrix, said method comprising the steps of:
identifying a continuous segment of genes having a segment length less than or equal to a predefined segment length as the subset of genes;
for each sample in the set of samples, projecting the DNA copy number data matrix on the sample and the subset of genes and forming a DNA copy number data column vector corresponding to each sample, respectively;
counting the number of values which are less than a predetermined threshold value in each of the data column vectors formed;
ordering the samples according to the counts of the respective DNA copy number vectors;
scoring order prefixes of the set of samples as to degree of deletion based on overabundance of values less than the predetermined threshold value in the corresponding DNA copy number submatrices relative to a corresponding complement DNA copy number submatrix, where the corresponding complement DNA copy number matrix contains measurements characterizing the same subset of genes as in the corresponding DNA copy number submatrix, but the complement of the subset of samples characterized in the corresponding DNA copy submatrix;
determining the maximum score from the degree of deletion scores; and
if the maximum score determined is greater than a predetermined significance threshold, concluding that the genomic-continuous submatrix corresponding to the subset of samples from which the maximum score was calculated, is a significantly deleted genomic-continuous submatrix.

47. The method of claim 46, wherein said predefined threshold value is zero.

48. The method of claim 46, wherein said order prefixes are scored using a binomial distribution function to score the overabundance of values less than the predetermined threshold value in the DNA copy number data submatrix relative to the number of values less than the predetermined threshold value in the entire DNA copy number data matrix using a binomial distribution function.

49. The method of claim 46, wherein said scoring comprises scoring the overabundance of values less than the predetermined threshold value in the DNA copy number data submatrix relative to the number of values less than the predetermined threshold value in the entire DNA copy number data matrix using a normal distribution function.

50. The method of claim 40, wherein said scoring comprises scoring the overabundance of genes in the subset of genes that have lower expression values for samples in the data submatrix than for samples in the complement data submatrix.

51. The method of claim 50, wherein said scoring comprises assigning a TNoM score to each gene in the subset of genes indicating its performance as a classifier of the subset of samples versus the complement of the subset of samples.

52. A system for co-analyzing DNA copy number data and gene expression data to identify significant relationships between alterations in genomic DNA and genes that are functionally effected by such alterations, comprising:
means for generating a gene expression data vector and a DNA copy number data vector for each gene in a set of genes for which DNA copy number data and gene expression data are provided across a plurality of samples;
means for selecting a gene expression data vector and determining correlation values between the selected gene expression data vector and DNA copy number vectors corresponding to the selected gene and genes in a defined chromosomal neighborhood of the selected gene, wherein the chromosomal neighborhood includes at least two genes.

53. A system for identifying chromosomal regions where consistently biased DNA copy number measurements and corresponding gene expression measurements correlate beyond an extent expected for the consistently biased DNA copy number measurements, comprising:
means for identifying a chromosomal neighborhood consisting of a set of loci located about a selected gene;
means for defining a simulation size by an integer L;
means for randomly drawing L−1 gene expression vectors from an expression data matrix having been generated by gene expression data measured across a plurality of samples;
means for computing a correlation of each randomly drawn gene expression vector to DNA copy number vectors having been generated by DNA copy number data across the plurality of samples for each of the respective genes in the chromosomal neighborhood identified in said identifying step;
means for ranking the computed correlation values computed with respect to the randomly drawn expression vectors, relative to a correlation value computed for the selected gene relative to the neighborhood of DNA copy number vectors; and
means for calculating an indicator of the degree of regional correlation of the DNA copy number vectors from the chromosomal neighborhood to the gene expression vector of the selected gene.

54. A system for detecting a chromosomal location in which a genomic aberration has occurred, samples that are affected by the genomic aberration, and the transcriptional effect of the aberration, based upon co-analysis of DNA copy number data and gene expression data wherein a DNA copy number data matrix provided contains DNA copy number measurements for a set of genes across a set of samples and a gene expression data matrix provided contains gene expression measurements for the same set of genes across the same samples, comprising:
means for identifying a genomic-continuous submatrix containing a subset of the set of genes measured to generate the DNA copy number data matrix and the gene expression data matrix, wherein the subset of the genes is a genomic-continuous set of genes, and wherein the genomic-continuous submatrix contains a subset of the set of samples measured to generate the DNA copy number data matrix and the gene expression data matrix;
means for projecting the DNA copy number data matrix and the gene expression data matrix on the subset of genes and subset of samples and respectively generating a DNA copy number data submatrix and a gene express data submatrix corresponding to the genomic-continuous submatrix; and
means for scoring the submatrices corresponding to the genomic-continuous submatrix relative to complement DNA copy number data and gene expression data submatrices corresponding to a complement submatrix defined by the same subset of genes in the genomic-continuous submatrix and a complement of the subset of samples in the genomic-continuous submatrix, to determine whether the genomic-continuous submatrix is significantly amplified or whether significant deletions have occurred in the genomic-continuous submatrix.

55. A system for identifying a high-scoring, significantly altered genomic-continuous submatrix, wherein each genomic-continuous submatrix contains a subset of a set of genes measured across a set of samples to generate a DNA copy number data matrix and a gene expression data matrix, wherein the subset of the genes is a genomic-continuous set of genes, and wherein each genomic-continuous submatrix contains a subset of the set of samples measured to generate the DNA copy number data matrix and the gene expression data matrix, comprising:
means for identifying a continuous segment of genes having a segment length less than or equal to a predefined segment length as the subset of genes;
for each sample in the set of samples, means for projecting the DNA copy number data matrix on the sample and the subset of genes and forming a DNA copy number data column vector corresponding to each sample, respectively;
means for counting the number of values which are greater than a predetermined threshold value in each of the data column vectors formed;
means for ordering the samples according to the counts of the respective DNA copy number vectors;
means for scoring order prefixes of the set of samples as to degree of amplification based on overabundance of positive values greater than the predetermined threshold value in the corresponding DNA copy number submatrices relative to a corresponding complement DNA copy number submatrix containing measurements characterizing the same subset of genes as in the corresponding DNA copy number submatrix, but the complement of the subset of samples characterized in the corresponding DNA copy submatrix;
means for determining the maximum score from the degree of amplification scores; and
means for concluding that the genomic-continuous submatrix corresponding to the subset of samples from which the maximum score was calculated is a significantly amplified genomic-continuous submatrix when the maximum score determined is greater than a predetermined significance threshold.

56. A system for identifying a high-scoring, significantly altered genomic-continuous submatrix, wherein each genomic-continuous submatrix contains a subset of a set of genes measured across a set of samples to generate a DNA copy number data matrix and a gene expression data matrix, wherein the subset of the genes is a genomic-continuous set of genes, and wherein each genomic-continuous submatrix contains a subset of the set of samples measured to generate the DNA copy number data matrix and the gene expression data matrix, comprising:
means for identifying a continuous segment of genes having a segment length less than or equal to a predefined segment length as the subset of genes;
for each sample in the set of samples, means for projecting the DNA copy number data matrix on the sample and the subset of genes and forming a DNA copy number data column vector corresponding to each sample, respectively;
means for counting the number of values which are less than a predetermined threshold value in each of the data column vectors formed;
means for ordering the samples according to the counts of the respective DNA copy number vectors;
means for scoring order prefixes of the set of samples as to degree of deletion based on overabundance of values less than the predetermined threshold value in the corresponding DNA copy number submatrices relative to a corresponding complement DNA copy number submatrix, where the corresponding complement DNA copy number matrix contains measurements characterizing the same subset of genes as in the corresponding DNA copy number submatrix, but the complement of the subset of samples characterized in the corresponding DNA copy submatrix;
means for determining the maximum score from the degree of deletion scores; and
means for concluding that the genomic-continuous submatrix corresponding to the subset of samples from which the maximum score was calculated, is a significantly deleted genomic-continuous submatrix, when the maximum score determined is greater than a predetermined significance threshold.

57. A computer readable medium carrying one or more sequences of instructions for co-analyzing DNA copy number data and gene expression data to identify significant relationships between alterations in genomic DNA and genes that are functionally effected by such alterations, wherein execution of one or more sequences of instructions by one or more processors causes the one or more processors to perform the steps of:
generating a gene expression data vector and a DNA copy number data vector for each gene in a set of genes for which DNA copy number data and gene expression data are provided across a plurality of samples;
selecting a gene expression data vector and determining correlation values between the selected gene expression data vector and DNA copy number vectors corresponding to the selected gene and genes in a defined chromosomal neighborhood of the selected gene, wherein the chromosomal neighborhood includes at least two genes.

58. A computer readable medium carrying one or more sequences of instructions for identifying chromosomal regions where consistently biased DNA copy number measurements and corresponding gene expression measurements correlate beyond an extent expected for the consistently biased DNA copy number measurements, wherein execution of one or more sequences of instructions by one or more processors causes the one or more processors to perform the steps of:
identifying a chromosomal neighborhood consisting of a set of loci located about a selected gene;
defining a simulation size by an integer L;
randomly drawing L−1 gene expression vectors from an expression data matrix having been generated by gene expression data measured across a plurality of samples;
computing a correlation of each randomly drawn gene expression vector to DNA copy number vectors having been generated by DNA copy number data across the plurality of samples for each of the respective genes in the chromosomal neighborhood identified in said identifying step;
ranking the computed correlation values computed with respect to the randomly drawn expression vectors, relative to a correlation value computed for the selected gene relative to the neighborhood of DNA copy number vectors; and
calculating an indicator of the degree of regional correlation of the DNA copy number vectors from the chromosomal neighborhood to the gene expression vector of the selected gene.

59. A computer readable medium carrying one or more sequences of instructions for detecting a chromosomal location in which a genomic aberration has occurred, samples that are affected by the genomic aberration, and the transcriptional effect of the aberration, based upon co-analysis of DNA copy number data and gene expression data wherein a DNA copy number data matrix provided contains DNA copy number measurements for a set of genes across a set of samples and a gene expression data matrix provided contains gene expression measurements for the same set of genes across the same samples, wherein execution of one or more sequences of instructions by one or more processors causes the one or more processors to perform the steps of:
identifying a genomic-continuous submatrix containing a subset of the set of genes measured to generate the DNA copy number data matrix and the gene expression data matrix, wherein the subset of the genes is a genomic-continuous set of genes, and wherein the genomic-continuous submatrix contains a subset of the set of samples measured to generate the DNA copy number data matrix and the gene expression data matrix;
projecting the DNA copy number data matrix and the gene expression data matrix on the subset of genes and subset of samples and respectively generating a DNA copy number data submatrix and a gene express data submatrix corresponding to the genomic-continuous submatrix; and
scoring the submatrices corresponding to the genomic-continuous submatrix relative to complement DNA copy number data and gene expression data submatrices corresponding to a complement submatrix defined by the same subset of genes in the genomic-continuous submatrix and a complement of the subset of samples in the genomic-continuous submatrix, to determine whether the genomic-continuous submatrix is significantly amplified or whether significant deletions have occurred in the genomic-continuous submatrix.

60. A computer readable medium carrying one or more sequences of instructions for identifying a high-scoring, significantly altered genomic-continuous submatrix, wherein each genomic-continuous submatrix contains a subset of a set of genes measured across a set of samples to generate a DNA copy number data matrix and a gene expression data matrix, wherein the subset of the genes is a genomic-continuous set of genes, and wherein each genomic-continuous submatrix contains a subset of the set of samples measured to generate the DNA copy number data matrix and the gene expression data matrix, wherein execution of one or more sequences of instructions by one or more processors causes the one or more processors to perform the steps of:
identifying a continuous segment of genes having a segment length less than or equal to a predefined segment length as the subset of genes;
for each sample in the set of samples, projecting the DNA copy number data matrix on the sample and the subset of genes and forming a DNA copy number data column vector corresponding to each sample, respectively;
counting the number of values which are greater than a predetermined threshold value in each of the data column vectors formed;
ordering the samples according to the counts of the respective DNA copy number vectors;
scoring order prefixes of the set of samples as to degree of amplification based on overabundance of values greater than the predetermined threshold value in the corresponding DNA copy number submatrices relative to a corresponding complement DNA copy number submatrix containing measurements characterizing the same subset of genes as in the corresponding DNA copy number submatrix, but the complement of the subset of samples characterized in the corresponding DNA copy submatrix;
determining the maximum score from the degree of amplification scores; and
concluding that the genomic-continuous submatrix corresponding to the subset of samples from which the maximum score was calculated is a significantly amplified genomic-continuous submatrix when the maximum score determined is greater than a predetermined significance threshold.

61. A computer readable medium carrying one or more sequences of instructions for identifying a high-scoring, significantly altered genomic-continuous submatrix, wherein each genomic-continuous submatrix contains a subset of a set of genes measured across a set of samples to generate a DNA copy number data matrix and a gene expression data matrix, wherein the subset of the genes is a genomic-continuous set of genes, and wherein each genomic-continuous submatrix contains a subset of the set of samples measured to generate the DNA copy number data matrix and the gene expression data matrix, wherein execution of one or more sequences of instructions by one or more processors causes the one or more processors to perform the steps of:
identifying a continuous segment of genes having a segment length less than or equal to a predefined segment length as the subset of genes;
for each sample in the set of samples, projecting the DNA copy number data matrix on the sample and the subset of genes and forming a DNA copy number data column vector corresponding to each sample, respectively;
counting the number of values which are less than a predetermined threshold value in each of the data column vectors formed;
ordering the samples according to the counts of the respective DNA copy number vectors;
scoring order prefixes of the set of samples as to degree of deletion based on overabundance of values less than the predetermined threshold value in the corresponding DNA copy number submatrices relative to a corresponding complement DNA copy number submatrix, where the corresponding complement DNA copy number matrix contains measurements characterizing the same subset of genes as in the corresponding DNA copy number submatrix, but the complement of the subset of samples characterized in the corresponding DNA copy submatrix;
determining the maximum score from the degree of deletion scores; and
concluding that the genomic-continuous submatrix corresponding to the subset of samples from which the maximum score was calculated, is a significantly deleted genomic-continuous submatrix, when the maximum score determined is greater than a predetermined significance threshold.
